(19) United States
(12) Patent Application Publication
(10) Pub. No.: US 2012/0283517 A1
(43) Pub. Date: Nov. 8, 2012

(54) Instrument handle for a medical diagnostic light, medical diagnostic light and medical diagnostic device

(76) Inventor: Regina Kirchner-Gottschalk, Markgroeningen (DE)

(21) Appl. No.: 13/462,857

(22) Filed: May 3, 2012

(30) Foreign Application Priority Data
May 5, 2011 (DE) 20 2011 050 045.4

(51) Int. Cl. A61B 1/06; A61B 3/12; A61B 1/227

(52) U.S. Cl. 600/200; 600/249

(57) ABSTRACT

An instrument handle (12) for a medical diagnostic light (10), to which a light instrument head (14) can be connected, accommodates or can be connected to an energy source. A manually operable light regulation device (20) is arranged in the instrument handle (12) at least partially behind a lateral wall opening (22) of the instrument handle (12) and is sealingly closed by a cover element (30) that is connected to the instrument handle (12) with material fit. The cover element (30) comprises an operating section (32) that can be manually pressed in and has markings (34) that can be haptically and optically differentiated, wherein the markings (34) are designed in the form of surface depressions and/or surface elevations of the cover element (30). A medical diagnostic light (10) uses the instrument handle (12) and a medical diagnostic device uses the diagnostic light (10).

This application claims Paris Convention priority of DE 20 2011 050 045.4 filed May 5, 2011 the complete disclosure of which is hereby incorporated by reference.

### BACKGROUND OF THE INVENTION

The invention concerns an instrument handle for a medical diagnostic light, a medical diagnostic light, and a medical diagnostic device.

Instrument handles of this type are used for medical diagnostic lights, such as e.g. ophthalmoscopes, otoscopes and/or dermatoscopes for medical examination of the ear, nose and throat region, the eyes or the skin.

The instrument handles are conventionally constructed in such a fashion that they have a coupling device at one end thereof for coupling to a light instrument head. The light instrument head may e.g. be an otoscope head or an ophthalmoscope head. Most of the light instrument heads have an integrated light source or are provided with light from a light source arranged in the instrument handle or from an external light source.

The instrument handles conventionally have a manually operable light regulation device for controlling the intensity of the light from the light source associated with the light instrument head. In the instrument handle, the light regulation device is often arranged behind a lateral wall opening of the instrument handle or extends therethrough to the outside such that the operator can operate the light regulation device with one finger or a thumb.

When the instrument handle is used on a patient, there is always the danger of soiling the instrument handle, in particular, with potentially infectious body fluids such as e.g. exudates and/or blood.

Cleaning of conventional instrument handles, in particular, in the area of the lateral wall opening thereof or also in their inside, can often only be realized to an unsatisfactory degree or only with undue difficulty.

It is the underlying purpose of the invention to improve an instrument handle for a medical diagnostic light and a medical diagnostic light comprising an instrument handle and a medical diagnostic device comprising a diagnostic light in such a fashion that they can be more easily cleaned.

### SUMMARY OF THE INVENTION

The object with respect to the instrument handle and the object with respect to the medical diagnostic light are achieved by the devices recited in the claims. The diagnostic device according to the teaching of the invention comprises the features in the associated dependent claim.

Advantageous further developments of the invention are the subject matter of the dependent claims.

The cover element that is connected to the instrument handle with material fit completely prevents entry of soiling matter, such as e.g. potentially infectious exudates or blood, into the instrument handle, in particular, in the area of the lateral wall opening of the instrument handle. For this reason, cleaning of the interior of the instrument handle is not necessary, which would otherwise be at least optionally required. The overall instrument handle can furthermore be cleaned/disinfected in a simplified fashion by spraying and/or wiping without thereby damaging the regulation device or other electrical or electronic components that are arranged in the instrument handle. This counteracts any possible danger of infection for an operator and increases the overall application safety of the instrument handle. If necessary, the instrument handle can also be inserted into a detergent or disinfectant, i.e. it can be completely immersed therein, which is advantageous, in particular, for heavy soiling or increased danger of contamination with germs.

In accordance with the invention, the cover element is injection-molded to the instrument handle and alternatively or additionally welded or glued to the instrument handle. In the first case, the instrument handle can be produced at particularly low cost. Welding of the cover element to the instrument handle moreover achieves a particularly high degree of sealing of the lateral wall opening of the instrument handle with respect to entry of particles or fluids. There is also no need to use adhesives, which is advantageous in view of material-specific recycling at a later point in time.

The inventive cover element also increases the operating convenience of the instrument handle, especially since the cover element improves the grip, i.e. improves the slip resistance as compared to conventional regulation devices with sliding switches or push-buttons which sometimes have sharp edges. The cover element also permits the instrument handle or the medical diagnostic light having such an instrument handle to be held in a particularly reliable fashion in a holding device, e.g. of a medical diagnostic device.

Projections on the instrument handle can furthermore be prevented and the surface of the instrument handle can be kept flat about its overall circumference.

The cover element advantageously has an operating section that can be manually pressed in, i.e. be adjusted or deformed in the direction of a longitudinal axis of the instrument handle. This enables convenient operation, in particular, of light push-buttons of the light regulation device with little force. The assignment of the functions on the light push-buttons can be selected and circuited differently in dependence on the respective application for the instrument handle.

In accordance with the invention, the operating section has markings that can be haptically and optically differentiated. For this reason, the operating functions of the regulation device covered by the cover element can be reliably, quickly and flawlessly detected. The overall operation of the instrument handle is thereby facilitated. The markings are designed in the form of surface depressions and/or elevations of the operating section. This is advantageous, in particular, when the user of the instrument handle wears gloves. Such markings are also not particularly susceptible to mechanical wear or corrosion by detergents or disinfectants.

In order to supply a light source arranged in the instrument handle or in a light instrument head that can be coupled to the instrument handle with electric operating energy, in accordance with the invention, the instrument handle is designed to accommodate an energy source such as e.g. an accumulator or a battery. The instrument handle may alternatively or additionally also be coupled or be able to be coupled to an electric line that is provided to be connected to an external energy source.

One particularly robust type of construction of the inventive instrument handle is characterized in that the cover element surrounds the overall circumference of the instrument handle. The cover element therefore surrounds the instrument handle like a cuff in that circumferential section of the instrument handle that is subjected to particular mechanical stress during operation. Undesired release of the cover element from the instrument handle is thereby reliably prevented.

In accordance with a preferred further development of the invention, the operating section of the cover element is delimited from a surface area of the cover element surrounding the operating area by a groove which is advantageously continuous and annularly surrounds the operating section. For this reason, the operating section can be detected in a simplified fashion (blindly) with the operating finger or a thumb.

In this connection, the operating section may additionally or alternatively also be curved inwardly towards the longitudinal axis of the instrument handle or be curved outwardly (concavely or convexly).

The cover element advantageously consists of a preferably synthetic elastic polymer. For this reason, the instrument handle can be held with particular reliability, and the regulation device or other current-carrying components within the instrument handle can moreover be electrically insulated to a particularly high degree with respect to the surroundings. This is advantageous in view of the operational safety of the instrument handle.

Embodiments of the invention are described in more detail below with reference to the drawing.

### BRIEF DESCRIPTION OF THE DRAWING

FIG. 1 shows a front view of an inventive ophthalmoscope for medical eye diagnostics, which has an inventive instrument handle;

FIG. 2 shows a side view of an otoscope with an instrument handle that corresponds to the instrument handle of FIG. 1;

FIG. 3 shows a side view of an otoscope with an instrument handle in accordance with FIG. 1; and

FIG. 4 shows a front view of an otoscopy and ophthalmoscopy system with the ophthalmoscope in accordance with FIG. 1 and the otoscope in accordance with FIG. 2.

### DESCRIPTION OF THE PREFERRED EMBODIMENT

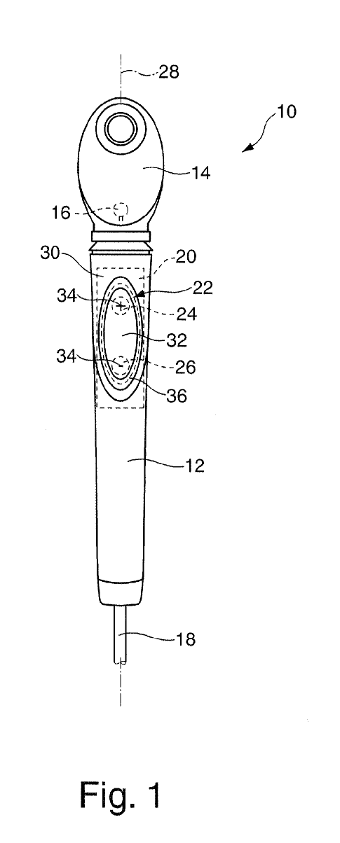

FIG. 1 shows a medical diagnostic light 10 designed as an ophthalmoscope for medical eye diagnostics. The diagnostic light 10 has an instrument handle 12 to one end of which a light instrument head 14, in the present case an ophthalmoscopy head, is detachably connected.

The light instrument head 14 has an electrically operated light source 16, in the present case a xenon halogen lamp or an LED, which is supplied with electric operating energy via an electric line 18 that can be connected to an external energy source and exits the instrument handle 12 at the other end.

In accordance with an embodiment of the invention, which is not shown in detail herein, the light source 16 may also be formed inside the instrument handle 12 or be designed in the form of an external light source 16 that can be coupled to the instrument handle 12.

A light regulation device 20, which is connected to the electric line 18 and to the light source 16, is provided for regulating the intensity of the light to be emitted by the light instrument head 14′, i.e. its light source 16.

The light regulation device 20 is provided in the instrument handle 12 partially behind a lateral wall opening 22 of the instrument handle 12, and has two light push-buttons 24, 26, which can be manually operated from the outside via the wall opening 22 of the instrument handle 12.

The two light push-buttons 24, 26 of the light regulation device 20 are arranged at a distance from each other along a longitudinal axis 28 of the instrument handle 12. The upper light push-button 24 in the figure is used for switching on the light source 16 and for increasing the intensity of the light emitted by the light source 16, whereas the lower light push-button 26 in the figure is used for decreasing or completely switching off the light source 16.

In order to protect the instrument handle 12 from entering soiling, such as e.g. particles and/or fluids (exudates, blood, etc.), the lateral wall opening 22 is sealingly closed by means of a cover element 30. The cover element 30 has an operating section 32 which is directly supported on the two light push-buttons 24, 26 and covers them from the outside. The operating section 32 of the cover element 30 can be pressed in towards the longitudinal axis 28 of the instrument handle 12 for operating the light push-buttons 24, 26 to such an extent with respect to the longitudinal axis 28 that the light push-buttons 24, 26 can be manually operated.

The operating section 32 has markings 34 that can be haptically and also visually differentiated in order to characterize the function of the light push-buttons 24, 26 arranged below the operating section 32. Each marking 34 is designed in the form of a depression, i.e. a recess in the surface of the operating section 32.

The operating section 32 is delimited from a surface area 38 of the cover element 28 surrounding the operating section 32 by a groove 36 that has an oval shape in the present embodiment. The groove 36 facilitates manual detection of the operating section 32 on the instrument handle 12 with an operating finger or a thumb. The groove 36 is moreover used for effectively (elastically) returning the operating section 32 of the cover element 30, which has been pressed in for operating the light regulation device 20, into its rest position illustrated here.

The cover element 30 is positively formed on the instrument handle 12 and surrounds it completely like a cuff. The cover element 30 is thereby molded to the instrument handle 12 and produced from an elastomer, i.e. an elastic polymer.

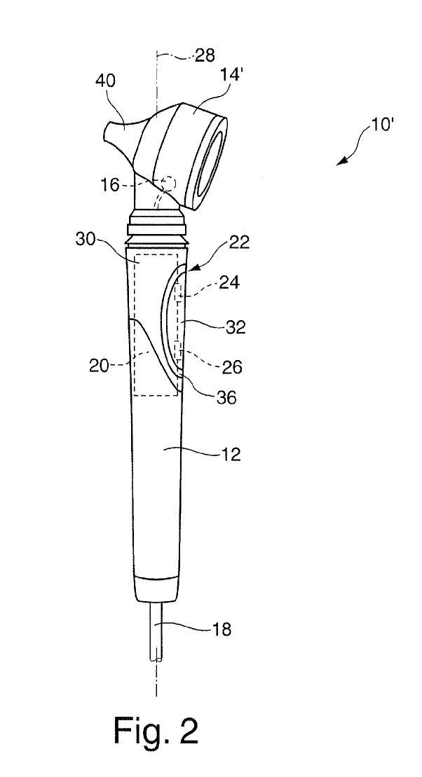

FIG. 2 shows a medical diagnostic light 10′ designed as an otoscope, comprising an instrument handle 12 as explained above. In contrast to the light instrument head 14 in accordance with FIG. 1, which is designed as ophthalmoscope head, the otoscope has a light instrument head 14′ which is designed as otoscope head and is conventionally provided with a tapered connecting fitting 40 for an ear specula which is not shown in detail. The figure clearly shows the cover element 30 that completely surrounds the instrument handle 12 on the peripheral side (like a cuff).

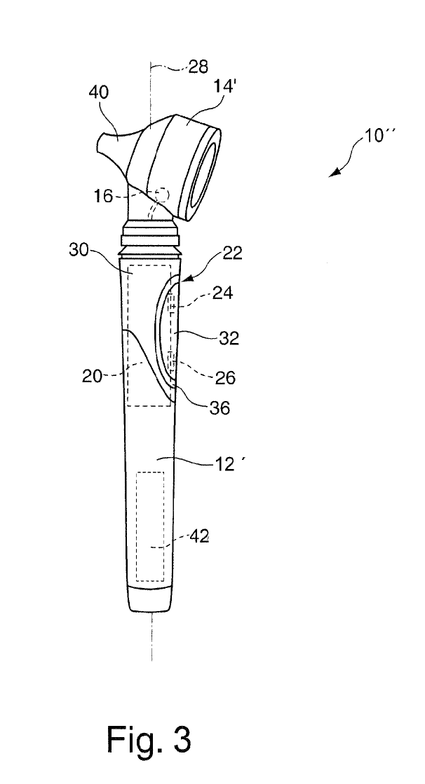

FIG. 3 shows a further embodiment of a medical diagnostic light 10″ designed as an otoscope, which differs from the above explained medical diagnostic light 10′ (FIG. 2) substantially in that it has an instrument handle 12′ that is provided for receiving an energy source 42, e.g. a battery, for supplying the light source 16 with electric operating energy.

For this reason, the diagnostic light 10″ has no electric line that can be connected to an external energy source, and can be operated as a portable hand-held device independently of an external energy source. The illustrated instrument handle 12′ can also be used for other medical diagnostic lights such as e.g. an ophthalmoscope and/or dermatoscope.

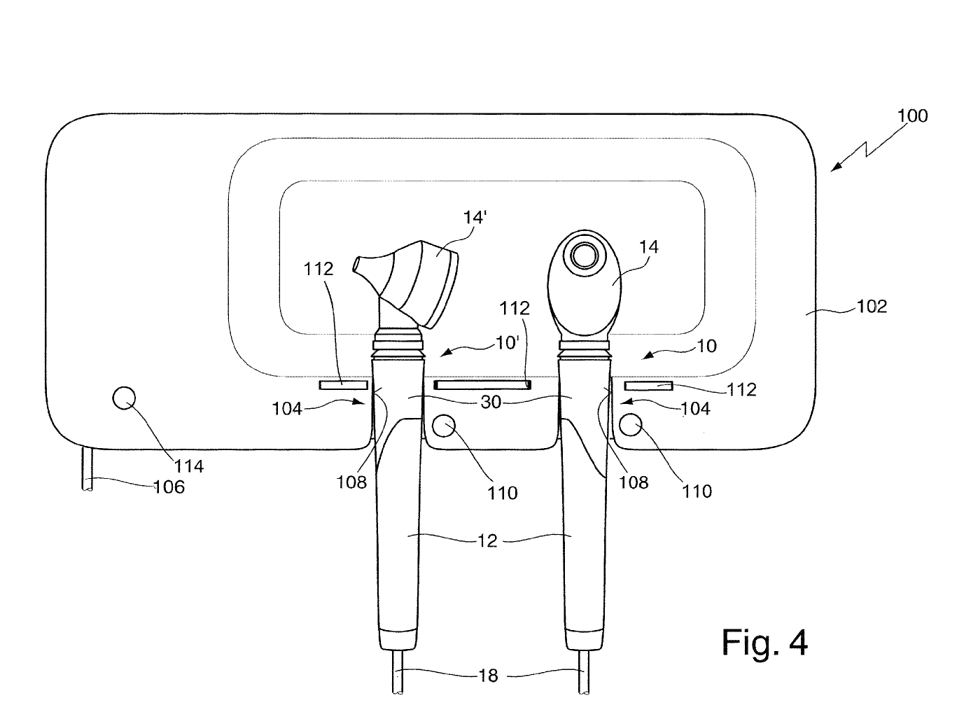

FIG. 4 shows a medical diagnostic device 100 with a housing 102 having two holding devices 104, each containing ready-to-use medical diagnostic lights 10, 10′, i.e. an ophthalmoscope (FIG. 1) and an otoscope (FIG. 2), as explained in connection with FIGS. 1 and 2.

The medical diagnostic device 100 has an electric connecting line 106 which can be coupled to an external energy source and can be electrically connected to each of the two medical diagnostic lights 10, 10′ via their electric lines 18 in a fashion that is not explained in more detail herein.

The cover elements 30 of the instrument handles of the medical diagnostic lights 10, 10′ rest against wall sections 108 of the holding devices 104 of the housing 102 and are held therein in a positive and frictionally engaging fashion (in particular due to the material properties of the cover elements 30) such that undesired inadvertent falling out of the otoscope 10′ or the ophthalmoscope 10 is reliably counteracted.

Each holding device 104 is associated with a magnetic switch 110 located inside the housing 102. The magnetic switches 110 automatically electrically connect the medical diagnostic lights 10, 10′ to the external electric energy source that is connected to the medical diagnostic device 100 and activate them when they are removed from the respective holding device 104, i.e. their light sources are switched on with a light intensity that can be freely predetermined if necessary. For this reason, the medical diagnostic lights 10, 10′ can be directly used.

The medical diagnostic device 100 moreover has induction devices 112 arranged in the housing 102, by means of which the (energy self-sufficient) diagnostic lights 10″ explained in connection with FIG. 3 and arranged in the holding device 104 can be inductively charged.

An optical display device 114, in the present case an LED, is used for optical information about the operational readiness or the (electric) operating status of the medical diagnostic device 100.

I claim:

1. An instrument handle for a medical diagnostic light, the handle comprising:
a first housing element, said first housing element structured for accepting a light instrument head;
a second housing element cooperating with said first housing element, said second housing element structured for accommodating an energy element;
a third housing element cooperating with said first and said second housing elements, said third housing element defining a lateral wall opening;
a manually operable light regulation device, said light regulation device disposed within the instrument handle at least partially behind said lateral wall opening, said light regulation device structured for regulating a light intensity of a light source associated with the light instrument head; and
a cover element, said cover element disposed, structured and dimensioned to sealingly close said lateral wall opening, said cover element connected to the instrument handle with material fit, wherein said cover element comprises an operating section that is structured to be manually pressed-in and has haptically and/or optically differentiated markings.

2. The instrument handle of claim 1, wherein said energy element is an energy source.

3. The instrument handle of claim 1, wherein said energy element is an electric line structured for coupling to an external energy source.

4. The instrument handle of claim 1, wherein said markings are depressions and/or elevations in a surface of said cover element.

5. The instrument handle of claim 1, wherein said cover element is at least one of injection-molded to the instrument handle, welded to the instrument handle and glued to the instrument handle.

6. The instrument handle of claim 1, wherein said cover element surrounds the instrument handle.

7. The instrument handle of claim 6, wherein said cover element surrounds the instrument handle around an overall circumference thereof.

8. The instrument handle of claim 1, wherein said operating section is delimited from a surface area of said cover element surrounding said operating area by a groove that annularly surrounds said operating section.

9. The instrument handle of claim 1, wherein said cover element comprises a synthetic polymer or an elastic synthetic polymer.

10. A medical diagnostic light, the light comprising:
the instrument handle of claim 1; and
a light instrument head connected to the instrument handle.

11. The medical diagnostic light of claim 10, wherein the light is an otoscope or an ophthalmoscope

12. A medical diagnostic device, the device comprising:
the medical diagnostic light of claim 10; and
an electric connecting line, said connecting line structured for connection to an electric energy source for supplying the medical diagnostic light with electric operating energy.
